(19) United States
(12) Patent Application Publication
(10) Pub. No.: US 2026/0201146 A1
(43) Pub. Date: Jul. 16, 2026

(54) KERATIN MATERIAL

(71) Applicant: WOOL RESEARCH ORGANISATION OF NEW ZEALAND INCORPORATED, Christchurch (NZ)

(72) Inventors: Robert James Mcclelland Kelly, Christchurch (NZ); Ben James Edwards, Rolleston (NZ); Gail Louise Krsinic, Springston (NZ); Alisa Dawn Roddick-Lanzilotta, Lincoln (NZ)

(73) Assignee: WOOL RESEARCH ORGANISATION OF NEW ZEALAND INCORPORATED, Christchurch (NZ)

(21) Appl. No.: 19/136,343

(22) PCT Filed: Dec. 22, 2023
(86) PCT No.: PCT/NZ2023/050152

§ 371 Date: Jun. 6, 2025

(30) Foreign Application Priority Data
Dec. 22, 2022 (AU) 2022903970

(51) Int. Cl. C08K 5/16; C07K 1/14; C07K 14/78; D04H 1/4266; D04H 1/4291; D04H 1/435; D04H 1/4382; D21H 13/24; D21H 13/34

(52) U.S. Cl. CPC C08K 5/16; C07K 1/14; C07K 14/78; D04H 1/4266; D04H 1/4291; D04H 1/435; D04H 1/43825; D04H 1/43835; D21H 13/24; D21H 13/34; D10B 2211/02; D10B 2321/021; D10B 2321/022; D10B 2331/04

(57) ABSTRACT

The present invention relates to a thermoset biopolymer derived from separated keratin fibre cellular components, a composite keratin material comprising the thermoset biopolymer, processes for the preparation thereof and uses thereof.

### TECHNICAL FIELD

The present invention relates to a thermoset biopolymer derived from separated keratin fibre cellular components, a composite keratin material comprising the thermoset biopolymer, processes for the preparation thereof and uses thereof.

### BACKGROUND ART

Fibrous proteins (also known as scleroproteins) are generally inert and insoluble in water. Fibrous proteins form long protein filaments shaped like rods or wires. They are structural or storage proteins. Fibrous proteins include keratin, collagen, elastin and fibroin.

Keratin fibres include wool, fur, hair and feathers. Wool is a keratin fibre produced by various animals including sheep, goats, camels and rabbits. The fibre structure comprises a cuticle, cortex, and medulla, although fine wools may lack the medulla.

The diameter of sheep wool typically ranges from about 10 microns to about 45 microns. Fibre diameter is an important characteristic of wool in relation to its quality and price. Finer wools are softer and suitable for use in garment manufacturing. There are a limited number of consumer applications remaining for stronger wool types such as flooring, bedding, upholstery, and hand knitting yarns.

Wool comprises three main histological components; two cellular components and a cell membrane complex that is present between the cells and maintains the structure together. The cellular components are cortical cells, which comprise the internal structure of the fibre, and cuticle cells, which overlap to form the outer layer. This complex biological assembly is created during wool growth by the body in the follicle.

A limitation of wool is that it is not a thermosetting material. While keratin is known to have a glass transition temperature, keratin fibres cannot effectively melt or process as a thermoplastic. Accordingly, the processing of wool has traditionally been limited by its shape and properties. Attempts to alter the shape and properties of wool have previously used wool in a fibrous form and relatively simple processes such as heat, steam or chemical setting of fibres.

The lack of ability to thermally process wool into a wide range of shapes and achieve full thermal transition has limited the application of wool outside of conventional fibre and textile products.

Accordingly, it is an object of the present invention to go some way to avoiding the above disadvantages; and/or to at least provide the public with a useful choice.

Other objects of the invention may become apparent from the following description which is given by way of example only.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date.

### SUMMARY OF THE INVENTION

In a first aspect, the present invention provides a thermoset biopolymer derived from separated keratin fibre cellular components.

In some embodiments, the thermoset biopolymer is obtained by subjecting the separated keratin fibre cellular components to elevated temperature and elevated pressure.

In a further aspect, the invention provides a composite keratin material comprising a thermoset biopolymer, wherein the thermoset biopolymer is derived from separated keratin fibre cellular components.

In some embodiments, the thermoset biopolymer is obtained by subjecting the separated keratin fibre cellular components to elevated temperature and elevated pressure.

In some embodiments, the composite keratin material further comprises cellulose fibres or polyethylene terephthalate (PET).

In a further aspect, the invention provides a process for preparing a composite keratin material comprising the steps of:
a) providing a mixture comprising separated keratin cellular fibre components, and
b) subjecting the mixture to elevated temperature and elevated pressure such that the separated keratin cellular fibre components form a thermoset biopolymer to provide the composite keratin material.

In some embodiments, the elevated temperature is at or above the glass transition temperature of the separated keratin fibre cellular components. In some embodiments, the elevated temperature is at least about 140° C. In some embodiments, the elevated temperature is at least about 160° C. In some embodiments, the elevated temperature is about 140° C. to about 220° C. In some embodiments, the elevated temperature is about 160° C. to about 200° C. In some embodiments, the elevated temperature is about 160° C.

In some embodiments, the elevated pressure is at least about 40 kN. In some embodiments, the elevated pressure is at least about 50 kN. In some embodiments, the elevated pressure is at least about 90 kN. In some embodiments, the elevated pressure is at least about 100 kN, about 150 kN, about 200 kN, about 250 kN, about 300 kN, about 350 kN, about 400 kN, about 450 kN, about 500 kN, about 550 kN, about 600 kN, about 650 kN, about 700 kN, about 750 kN, about 800 kN, about 850 kN or about 900 kN. In some embodiments, the elevated pressure is at least about 450 kN. In some embodiments, the elevated pressure is at least about 900 kN.

In some embodiments, the mixture is subjected to the elevated temperature and elevated pressure for a period of at least about 1 minute. In some embodiments, the keratin mixture is subjected to the elevated temperature and elevated pressure for a period of at least about 1 minute, about 2 minutes or about 3 minutes. In some embodiments, the keratin mixture is subjected to the elevated temperature and elevated pressure for a period of about 3 minutes.

In some embodiments, the elevated temperature and elevated pressure are achieved by hot-pressing the mixture. In some embodiments, the mixture is hot-pressed between metal plates.

In some embodiments, the mixture in step (a) is provided as a sheet. In some embodiments, the mixture in step (a) is provided as a layer of two or more sheets, optionally a layer of 2 to 10 sheets or 2 to 5 sheets. In some embodiments, each sheet is formed by pressing a slurry comprising separated keratin cellular fibre components. In some embodiments, each sheet is couched, pressed and dried.

In some embodiments, the mixture is subjected to elevated temperature and elevated pressure for a time sufficient to provide a material having at least one of the following properties:
i) a tensile strength of at least about 30 Nm/g,
ii) an air resistance of at least about 10 s/100 mL,
iii) a Cobb₃₀ value below about 100 g/m².

In some embodiments, the mixture further comprises a cellulose material. In some embodiments, the cellulose material is derived from a plant or plant parts. In some embodiments, the cellulose material is derived from a plant or plant parts selected from the group consisting of wood, cotton, corn, flax, jute, ramie, straw, hemp, bagasse, miscanthus and a combination of any two or more thereof. In some embodiments, the wood is wood pulp or wood fibre.

In some embodiments, the mixture further comprises a synthetic fibre. In some embodiments, the synthetic fibre comprises a polymer selected from the group consisting of a polyester (e.g. a polyethylene terephthalate, PET), a polyacrylic, a polychloroprene (e.g. neoprene), a polyolefin, a polyurethane (e.g. spandex), a polyamide (e.g. nylon) and a combination of any two or more thereof. In some embodiments, the synthetic fibre is a polyethylene terephthalate.

In some embodiments, the mixture further comprises water.

In some embodiments, the mixture further comprises an additive selected from the group consisting of a reducing agent, a plasticiser, an oil and a combination of any two or more thereof.

In some embodiments, the mixture in step (a) is provided as a sheet or layer of sheets and the additive is applied to the sheet or layer of sheets before step (b), and optionally wherein the additive is applied by immersing the sheet or layer of sheets in the additive, brushing the additive onto the sheet or layer of sheets, or spraying the additive on the sheet or layer of sheets. In some embodiments, the additive is applied to each sheet before forming the layer of sheets.

In some embodiments, the reducing agent is selected from the group consisting of a sulfite, a metabisulfite, a sulfide, a thioglycolate, cysteine and a combination of any two or more thereof. In some embodiments, the reducing agent is selected from the group consisting of sodium sulfite, sodium metabisulfite, sodium sulfide, sodium thioglycolate, cysteine and a combination of any two or more thereof. In some embodiments, the reducing agent is sodium sulfite.

In some embodiments, the plasticiser is selected from glycerol or polypropylene glycol. In some embodiments, the glycerol is an aqueous glycerol solution, a 1-50 w/w % aqueous glycerol solution, a 10-50 w/w % aqueous glycerol solution or a 50 w/w % aqueous glycerol solution.

In some embodiments, the oil is an oil with a flashpoint higher than about 155° C. In some embodiments, the oil is an oil with a smoke point higher than about 140° C. In some embodiments, the oil is a natural oil or a synthetic oil. In some embodiments, the natural oil is a vegetable oil or animal-derived oil. In some embodiments, the vegetable oil is almond oil, avocado oil, coconut oil, palm oil, peanut oil, canola oil, safflower oil, sesame oil, soyabean oil, sunflower oil, grapeseed oil. In some embodiments, the synthetic oil is silicone oil.

In some embodiments, the composite keratin material has one or more of the following properties:
i) a tensile strength of at least about 30 Nm/g,
ii) an air resistance of at least about 10 s/100 mL,
iii) a Cobb₃₀ value below about 100 g/m².

In some embodiments, the separated keratin fibre cellular components are obtained from a source selected from the group consisting of wool, hair, fur, feathers and a combination of any two or more thereof. In some embodiments, the separated keratin fibre cellular components are obtained from wool. In some embodiments, the separated keratin fibre cellular components are obtained from sheep wool.

In some embodiments, the composite keratin material further comprises a cellulose material. In some embodiments, the cellulose material is derived from a plant or plant parts. In some embodiments, the cellulose material is derived from a plant or plant parts selected from the group consisting of wood, cotton, corn, flax, jute, ramie, straw, hemp, bagasse, miscanthus and a combination of any two or more thereof. In some embodiments, the wood is wood pulp or wood fibre.

In some embodiments, the composite keratin material further comprises a synthetic fibre. In some embodiments, the synthetic fibre comprises a polymer selected from the group consisting of a polyester (e.g. a polyethylene terephthalate, PET), a polyacrylic, a polychloroprene (e.g. neoprene), a polyolefin, a polyurethane (e.g. spandex), a polyamide (e.g. nylon) and a combination of any two or more thereof. In some embodiments, the synthetic fibre is a polyethylene terephthalate.

In some embodiments, the composite keratin material comprises keratin fibre cellular components and a cellulose material in a weight ratio of about 5:95 to about 95:5. In some embodiments, the composite keratin material comprises keratin fibre cellular components and a cellulose material in a weight ratio of about 10:90 to about 90:10, about 20:80 to about 80:20, about 30:70 to about 70:30, about 40:60 to about 60:40 or about 45:55 to about 55:45. In some embodiments, the composite keratin material comprises keratin fibre cellular components and a cellulose material in a weight ratio of about 30:70 to about 70:30, about 40:60 to about 60:40 or about 45:55 to about 55:45.

In some embodiments, the composite keratin material has at least two of properties i) to iii). In some embodiments, the composite keratin material has properties i) to iii).

In some embodiments, the composite keratin material has a tensile strength of at least about 35 Nm/g, about 36 Nm/g, about 37 Nm/g, about 38 Nm/g, about 39 Nm/g, about 40 Nm/g, about 41 Nm/g, about 42 Nm/g, about 43 Nm/g, about 44 Nm/g, about 45 Nm/g, about 46 Nm/g, about 47 Nm/g, about 48 Nm/g, about 49 Nm/g, about 50 Nm/g, about 51 Nm/g, about 52 Nm/g, about 53 Nm/g, about 54 Nm/g or about 55 Nm/g. In some embodiments, the composite keratin material has a tensile strength of at least about 40 Nm/g. In some embodiments, the composite keratin material has a tensile strength of at least about 45 Nm/g. In some embodiments, the composite keratin material has a tensile strength of at least about 50 Nm/g. In some embodiments, the composite keratin material has a tensile strength of at least about 55 Nm/g.

In some embodiments, the composite keratin material has an air resistance of at least about 20 s/100 mL, about 30 s/100 mL, about 40 s/100 mL, about 50 s/100 mL, about 60 s/100 mL, about 70 s/100 mL, about 80 s/100 mL, about 90 s/100 mL, about 100 s/100 mL, about 110 s/100 mL, about 120 s/100 mL or about 130 s/100 mL. In some embodiments, the composite keratin material has an air resistance of at least about 50 s/100 mL.

In some embodiments, the composite keratin material has a Cobb₃₀ value below about 90 g/m², about 80 g/m², about 70 g/m², about 60 g/m², about 50 g/m², about 45 g/m² or about 40 g/m².

In some embodiments, the composite keratin material is in the form of a sheet.

In some embodiments, the sheet has a thickness greater than 100 μm, greater than 150 μm, greater than 200 μm, or greater than 250 μm.

In another aspect, the invention provides a composite keratin material prepared by a process according to the invention.

In a further aspect, the invention provides a paper comprising the composite keratin material according to the invention.

In a further aspect, the invention provides an artificial leather comprising the composite keratin material according to the invention.

In a further aspect, the invention provides a fabric comprising the composite keratin material according to the invention.

In some embodiments, the composite keratin material according to the invention is a paper, a fabric or an artificial leather.

In some embodiments, the fabric is a non-woven fabric.

In another aspect, the invention relates to the use of the composite keratin material as a plastic substitute.

In another aspect, the invention relates to the use of the composite keratin material as a substitute for a chemical binder in the preparation of a synthetic non-woven textile.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

In addition, where features or aspects of the invention are described in terms of Markush groups, those persons skilled in the art will appreciate that the invention is also thereby described in terms of any individual member or subgroup of members of the Markush group.

As used herein “(s)” following a noun means the plural and/or singular forms of the noun.

As used herein the term “and/or” means “and” or “or” or both.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

The term “keratin fibre cellular components” as used in this specification means keratin fibre cuticle cells, keratin fibre cortical cells, or a combination of keratin fibre cuticle and cortical cells. The separated keratin fibre cellular components are provided as separated cells that are physically distinct from each other. This is different to native keratin fibres where cuticle cells and cortical cells are bound together in a specific ordered arrangement determined by the growth of the fibre through a follicle. The keratin fibre cellular components may be prepared by a method disclosed in WO 2020/080961 A1, which is hereby incorporated by reference in its entirety. The wool cortical cells typically have an ellipsoid shape (preferably spindle shape) and are typically 50-150 μm long (preferably 70-120 μm) with a diameter of 1-10 μm (preferably 4-8 μm). The wool cuticle cells are typically irregular shape with dimensions of 1×10-15×15-40 μm. Preferably, the keratin fibre cellular components are a combination of keratin fibre cuticle and cortical cells.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

Although the present invention is broadly as defined above, those persons skilled in the art will appreciate that the invention is not limited thereto and that the invention also includes embodiments of which the following description gives examples.

### BRIEF DESCRIPTION OF THE DRAWINGS

The invention will now be described with reference to the Figures in which:

FIG. 1 shows the effect of hot-pressing treatments on tensile strength.

FIG. 2 shows the effect of hot-pressing force on air resistance for 45% wool cells: 55% wood fibre handsheets wet pressed at 160° C. for 3 minutes.

FIG. 3 shows the effect of pressing force on Cobb water absorption results for 45% wool cells: 55% wood fibre paper wet pressed at 160° C. for 3 minutes.

FIG. 4 shows the effect of pressing force on air resistance for unpressed and pressed wood fibre and wool cells:wood fibre mix sheets.

FIG. 5 shows the effect of adding wool fibre to wood fibre and wet pressing on handsheet Cobb water absorbance.

FIG. 6 shows the stain area (mm²) for wool cells:wood fibre mix handsheets and 100% wood fibre handsheets.

FIG. 7 shows a wetlaid nonwoven fabric comprising PET and melded wool cells in a weight ratio of 60:40.

FIG. 8 shows a wetlaid nonwoven fabric comprising PET and melded wool cells in a weight ratio of 80:20.

FIG. 9 shows a wetlaid nonwoven fabric comprising PET and melded wool cells in a weight ratio of 90:10.

FIG. 10 shows the liquid absorption capacity of wetlaid nonwoven PET fabrics with and without melded wool cells (SC).

FIG. 11 shows the liquid wicking rate of wetlaid nonwoven PET fabrics with and without melded wool cells (SC).

FIG. 12 shows tensile strength of wetlaid nonwoven PET fabrics with and without melded wool cells (SC).

### DETAILED DESCRIPTION OF THE INVENTION

The present invention relates to a surprising discovery that separated keratin fibre cellular components form a thermoset biopolymer when subjected to elevated temperature and elevated pressure. The thermoset biopolymer is useful in the preparation of a composite keratin material, e.g., comprising a cellulose material or a synthetic fibre. Advantageously, the composite keratin material may have improved properties, e.g. tensile strength or barrier properties, relative to the same material without the thermoset biopolymer. Importantly, this effect is not observed in comparable materials prepared with keratin fibres instead of the separated keratin fibre cellular components. Additionally, the thermosetting properties of the thermoset biopolymer allow it to be, e.g. formed into various shapes, unlike keratin fibres.

The thermoset biopolymer is prepared by subjecting a mixture comprising the separated keratin fibre cellular components to elevated temperature and elevated pressure. In some embodiments, the elevated temperature may be selected based on the glass transition temperature of the keratin cellular fibre components. For example, the keratin mixture may be subjected to a temperature at or above the glass transition temperature of the separated keratin fibre cellular components. In some embodiments, the keratin mixture may be subjected to a temperature of at least about 140° C. In some embodiments, the keratin mixture may be subjected to a temperature of about 160° C. Those persons skilled in the art will appreciate that the temperature needed to form the thermoset biopolymer may be influenced by the pressure. For example, at higher pressures, a lower temperature may be needed to form the thermoset biopolymer.

For example, the mixture may be subjected to a pressure of at least about 40 kN. In some embodiments, the keratin mixture may be subjected to a pressure of at least about 50 kN, about 90 kN, about 100 kN, about 150 kN, about 200 kN, about 250 kN, about 300 kN, about 350 kN, about 400 kN, about 450 kN, about 500 kN, about 550 kN, about 600 kN, about 650 kN, about 700 kN, about 750 kN, about 800 kN, about 850 kN or about 900 kN. In some embodiments, the keratin mixture may be subjected to a pressure of at least about 450 kN. In some embodiments, the keratin mixture may be subjected to a pressure of at least about 900 kN.

In some embodiments, the mixture is subjected to the elevated temperature and elevated pressure for a period of at least about 1 minute. In some embodiments, the mixture is subjected to the elevated temperature and elevated pressure for a period of about 3 minutes.

In some embodiments, the mixture is subjected to a temperature of at least about 160° C. and a pressure of at least about 40 kN for a period of about 3 minutes. In some embodiments, the mixture is subjected to a temperature of at least about 160° C. and a pressure of at least about 90 kN for a period of about 3 minutes. In some embodiments, the mixture is subjected to a temperature of at least about 160° C. and a pressure of at least about 450 kN for a period of about 3 minutes. In some embodiments, the mixture is subjected to a temperature of at least about 160° C. and a pressure of at least about 900 KN for a period of about 3 minutes. Those persons skilled in the art may select the appropriate combination of temperature, pressure and time to obtain the thermoset biopolymer or composite keratin material.

The mixture may be subjected to elevated temperature and elevated pressure by, e.g., hot-pressing the mixture. In some embodiments, the mixture is hot-pressed between metal plates.

To achieve the desired pressure, the mixture may be pressed between two plates, e.g. two metal plates. For example, the hot-pressing step may be performed in Siempelkamp press.

The present description is substantially directed to keratin fibre cellular components obtained from wool. However, the invention is not limited thereto and cellular components obtained from other keratin fibres, such as wool, hair, fur and feathers, are also useful in the present invention. In a preferred embodiment, the keratin fibres are wool, hair, or fur, or a mixture of any two or more thereof. In a preferred embodiment, the wool is sheep wool.

The keratin fibre cellular components of the present invention may be prepared by methods known to those persons skilled in the art. For example, the keratin fibre cellular components may be prepared by a method as disclosed in WO 2020/080961 A1.

The mixture may further comprise a cellulose material. The cellulose material may be derived from a plant or plant parts, e.g., wood, cotton, corn, flax, jute, ramie, straw, hemp, bagasse, miscanthus or a combination of any two or more thereof. In some embodiments, the wood is wood pulp or wood fibre. Those persons skilled in the art will appreciate other sources of cellulose, particularly cellulose fibres, may be useful in the invention disclosed herein.

The mixture may comprise keratin fibre cellular components and a cellulose material in a weight ratio of about 5:95 to about 95:5, e.g., about 10:90 to about 90:10, about 20:80 to about 80:20, about 30:70 to about 70:30, about 40:60 to about 60:40 or about 45:55 to about 55:45. In some embodiments, the mixture comprises keratin fibre cellular components and a cellulose material in a weight ratio of about 30:70 to about 70:30, about 40:60 to about 60:40 or about 45:55 to about 55:45. The weight ratio of keratin fibre cellular components to the cellulose material may be selected based on the desired application of the resulting material. For example, a paper comprising composite keratin material may be prepared from keratin mixture comprising keratin fibre cellular components and a cellulose material in a weight ratio of about 45:55 to about 55:45.

Accordingly, the composite keratin material may comprise keratin fibre cellular components and the cellulose material in a weight ratio of about 5:95 to about 95:5, e.g., about 10:90 to about 90:10, about 20:80 to about 80:20, about 30:70 to about 70:30, about 40:60 to about 60:40 or about 45:55 to about 55:45. In some embodiments, the composite keratin material comprises keratin fibre cellular components and the cellulose material in a weight ratio of about 30:70 to about 70:30, about 40:60 to about 60:40 or about 45:55 to about 55:45.

The composite keratin material may comprise a synthetic fibre. Suitable synthetic fibres includes, but are not limited to, those fibres made from a polymer selected from the group consisting of a polyester (e.g. a polyethylene terephthalate, PET), a polyacrylic, a polychloroprene (e.g. neoprene), a polyolefin, a polyurethane (e.g. spandex), a polyamide (e.g. nylon) and a combination of any two or more thereof.

For example, the mixture may comprise keratin fibre cellular components and the synthetic fibre in a weight ratio of about 5:95 to about 75:25, about 10:90 to about 70:30, e.g., about 15:85 to about 65:35, about 20:80 to about 60:40, about 25:75 to about 65:45, about 30:70 to about 50:50 or about 35:65 to about 45:55. In some embodiments, the mixture comprises keratin fibre cellular components and the synthetic fibre in a weight ratio of about 30:70 to about 70:30 or about 35:65 to about 45:55. The weight ratio of keratin fibre cellular components to the PET may be selected based on the desired application of the resulting material. For example, a fabric comprising composite keratin material may be prepared from keratin mixture comprising keratin fibre cellular components and the synthetic fibre in a weight ratio of about 40:60.

Accordingly, the composite keratin material may comprise keratin fibre cellular components and the synthetic fibre in a weight ratio of about 5:95 to about 75:25, about 10:90 to about 70:30, e.g., about 15:85 to about 65:35, about 20:80 to about 60:40, about 25:75 to about 65:45, about 30:70 to about 50:50 or about 35:65 to about 45:55. In some embodiments, the composite keratin material comprises keratin fibre cellular components and the synthetic fibre in a weight ratio of about 20:80 to about 60:40, about 25:75 to about 65:45, about 30:70 to about 50:50 or about 35:65 to about 45:55.

The mixture may further comprise an additive such as a reducing agent, a plasticiser, an oil or a combination of any two or more thereof.

Suitable reducing agents include, but are not limited to, a sulfite, a metabisulfite, a sulfide, a thioglycolate, cysteine and a combination of two or more thereof. The reducing agent may be a salt, e.g. a sodium salt or a potassium salt. Accordingly, in some embodiments, the reducing agent is selected from the group consisting of sodium sulfite, sodium metabisulfite, sodium sulfide, sodium thioglycolate, cysteine and a combination of two or more thereof. Without wishing to be bound by theory, it is believed the reducing agent may reduce disulfide bonds in the separated keratin fibre cellular components resulting in increased bonding when the components are subjected to elevated temperature and elevated pressure. Advantageously, the reducing agent may improve the material properties, e.g. grease resistance, of the resulting thermoset biopolymer or composite keratin material.

Suitable plasticisers include, but are not limited to glycerol and propylene glycol. For example, the glycerol may be provided as an aqueous glycerol solution, e.g., a 1-50 w/w % aqueous glycerol solution or 10-50 w/w % aqueous glycerol solution. Suitable oils include, but are not limited to a natural oil (such as a vegetable oil or animal-derived oil) or a synthetic oil. In some embodiments, the vegetable oil is almond oil, avocado oil, coconut oil, palm oil, peanut oil, canola oil, safflower oil, sesame oil, soyabean oil, sunflower oil, grapeseed oil. In some embodiments, the synthetic oil is silicone oil. Generally, it is expected that suitable oils will have a flashpoint higher than about 155° C. and/or a smoke point higher than about 140° C. Advantageously, the plasticiser and/or oil may improve the flexibility of the resulting thermoset biopolymer or composite keratin material.

The mixture may comprise further additives. Those persons skilled in the art will appreciate that conventional additives known in the art may be useful based on the intended product and application. For example, the mixture may comprise a dispersion agent (such as Pluronic F-108, a poly(ethylene glycol)-block-poly(propylene glycol)-block-poly(ethylene glycol) polymer) and/or a defoaming agent (such as Tetronic 90R4, ethylenediamine tetrakis(ethoxylate-block-propoxylate) tetrol). Advantageously, such agents may improve the homogeneity and dispersion of the mixture.

The keratin mixture may be formed into a sheet before being subjected to elevated temperature and elevated pressure. The handsheet may be formed by conventional techniques known in the art, e.g. based on Australian Pulp and Paper Technical Association (Appita) standard method AS/NZS 1301.203s: 2017 or TAPPI standard T205.

For example, the sheet may be formed by preparing a slurry comprising keratin fibre cellular components in a liquid, such as water. The slurry may be prepared by dispersing a dry keratin fibre cellular component material in the liquid. When the keratin mixture further comprises a cellulose material and/or a synthetic fibre, the keratin mixture may be prepared by dispersing a dry keratin fibre cellular component material and a dry cellulose material and/or the synthetic fibre in the liquid either together or into separate slurries that are subsequently mixed. Preparation of the slurry may include a disintegration step to disperse the materials. Optionally, the dry keratin fibre cellular component material and/or the dry cellulose material and/or the synthetic fibre may be refined before forming into a slurry.

The slurry is then formed into a sheet, e.g. by transferring the slurry into a sheet machine and at least partially removing the liquid (such as water). The sheet then may be subjected to elevated temperature and elevated pressure without any further processing to obtain the composite keratin material. Alternatively, the sheet may be subject to one or more processing steps before being subjected to elevated temperature and elevated pressure, including but not limited to, crouching, pressing, drying and/or conditioning. These further processing steps may be conventional techniques known in the art, as described in TAPPI standard T205.

When the mixture comprises an additive, the additive may be added to the slurry or applied to the sheet. For example, the additive may be applied to the sheet by immersing the sheet in the additive, brushing the additive onto the sheet, or spraying the additive onto the sheet. In embodiments where a layer of sheets is hot pressed, the additive may be applied to the layer of sheets, or to each sheet before forming the layer of sheets.

Optionally, the sheet may be washed, e.g., with water or a dilute acid (such as 2 w/w % sulfuric acid) prior to the hot-pressing step. The washed sheet is then optionally dried and pressed again prior to the hot-pressing step.

In some embodiments, the keratin mixture consists of the keratin cellular fibre components and the cellulose material. In some embodiments, the keratin mixture consists essentially of the keratin cellular fibre components and the cellulose material. In some embodiments, the keratin mixture consists of the keratin cellular fibre components, the cellulose material and water. In some embodiments, the keratin mixture consists essentially of the keratin cellular fibre components, the cellulose material and water. In some embodiments, the keratin mixture consists of the keratin cellular fibre components, the cellulose material, water and sodium sulfite. In some embodiments, the keratin mixture consists essentially of the keratin cellular fibre components, the cellulose material, water and sodium sulfite.

In some embodiments, the keratin mixture consists of the keratin cellular fibre components and the synthetic fibre. In some embodiments, the keratin mixture consists essentially of the keratin cellular fibre components and the synthetic fibre. In some embodiments, the keratin mixture consists of the keratin cellular fibre components, the synthetic fibre and water. In some embodiments, the keratin mixture consists essentially of the keratin cellular fibre components, the synthetic fibre and water. In some embodiments, the keratin mixture consists of the keratin cellular fibre components, the synthetic fibre, water and sodium sulfite. In some embodiments, the keratin mixture consists essentially of the keratin cellular fibre components, the synthetic fibre, water and sodium sulfite.

The composite keratin material may be in the form of a sheet. The sheet may be prepared in various thicknesses. The desired thickness may be selected, e.g., based on the intended use of the sheet. For example, a sheet comprising the composite keratin material intended for use as paper may have a thickness of about 50 μm to about 200 μm. In some embodiments, the sheet comprising the composite keratin material has a thickness of about 75 μm to about 175 μm, about 90 μm to about 150 μm or about 100 μm to about 130 μm. In some embodiments, the sheet comprising the composite keratin material has a thickness of about 90 μm to about 110 μm. In some embodiments, the sheet comprising the composite keratin material has a thickness of about 100 μm to about 130 μm. For example, the sheet comprising the composite keratin material may have a thickness of about 80 μm, about 85 μm, about 90 μm, about 95 μm, about 100 μm, about 105 μm, about 100 μm, about 105 μm, about 110 μm, about 115 μm, about 120 μm, about 125 μm, about 130 μm, about 135 μm or about 140 μm. A sheet comprising the composite keratin material intended for use as paper may have a thickness of greater than about 100 μm, greater than about 150 μm, greater than about 200 μm or greater than 250 μm.

The sheet may also be provided in various weights. For example, the sheet comprising the composite keratin material may have a weight of about 50 g/m² to about 150 g/m². In some embodiments, the sheet has a weight of about 60 g/m² to about 140 g/m², about 70 g/m² to about 130 g/m², about 80 g/m² to about 120 g/m² or about 90 g/m² to about 110 g/m². In some embodiments, the sheet has a weight of about 80 g/m², about 90 g/m², about 100 g/m² or about 110 g/m². In some embodiments, the sheet has a weight of about 100 g/m².

Advantageously, the composite keratin material may have one or more improved properties, e.g., improved tensile strength or improved barrier properties, relative to the same material without the thermoset biopolymer. The barrier properties may include resistance to air, water and/or grease.

The composite keratin material may have an improved tensile strength compared to a similar material that does not contain the thermoset biopolymer derived from separated keratin fibre cellular components. For example, the composite keratin material may have a tensile strength of at least about 30 Nm/g, e.g., at least about 35 Nm/g, about 40 Nm/g, about 45 Nm/g, about 50 Nm/g, about 51 Nm/g, about 55 Nm/g, about 60 Nm/g, about 65 Nm/g, about 70 Nm/g, about 75 Nm/g, about 80 Nm/g, about 85 Nm/g, about 90 Nm/g, about 95 Nm/g, about 100 Nm/g, about 105 Nm/g, about 110 Nm/g, about 115 Nm/g, about 120 Nm/g, about 125 Nm/g, about 130 Nm/g, about 135 Nm/g, about 140 Nm/g, about 145 Nm/g, about 150 Nm/g, about 155 Nm/g or about 160 Nm/g. In some embodiments, the composite keratin material has a tensile strength of at least about 40 Nm/g, about 45 Nm/g, about 50 Nm/g or about 55 Nm/g. In some embodiments, the composite keratin material comprises the thermoset biopolymer and cellulose material and has a tensile strength of at least about 30 Nm/g, e.g., at least about 35 Nm/g, about 36 Nm/g, about 37 Nm/g, about 38 Nm/g, about 39 Nm/g, about 40 Nm/g, about 41 Nm/g, about 42 Nm/g, about 43 Nm/g, about 44 Nm/g, about 45 Nm/g, about 46 Nm/g, about 47 Nm/g, about 48 Nm/g, about 49 Nm/g, about 50 Nm/g, about 51 Nm/g, about 52 Nm/g, about 53 Nm/g, about 54 Nm/g or about 55 Nm/g. In some embodiments, the composite keratin material comprises the thermoset biopolymer and a synthetic fibre (e.g. a PET fibre) and has a tensile strength of at least about 100 Nm/g, about 105 Nm/g, about 110 Nm/g, about 115 Nm/g, about 120 Nm/g, about 125 Nm/g, about 130 Nm/g, about 135 Nm/g, about 140 Nm/g, about 145 Nm/g, about 150 Nm/g, about 155 Nm/g or about 160 Nm/g. In some embodiments, the composite keratin material comprises the thermoset biopolymer and a synthetic fibre (e.g. a PET fibre) and has a tensile strength of at least about 140 Nm/g, about 145 Nm/g, about 150 Nm/g or about 155 Nm/g. Tensile strength may be measured by conventional techniques known in the art, such as “Tensile properties of paper and paperboard”, Test Method T 494 2013.

The composite keratin material may an improved Cobb₃₀ value compared to a similar material that does not contain the thermoset biopolymer derived from separated keratin fibre cellular components. For example, the composite keratin material may a Cobb₃₀ value below about 90 g/m², about 80 g/m², about 70 g/m², about 60 g/m², about 50 g/m², about 45 g/m² or about 40 g/m². In some embodiments, the composite keratin material comprises the thermoset biopolymer and cellulose material and a Cobb₃₀ value below about 90 g/m², about 80 g/m², about 70 g/m², about 60 g/m², about 50 g/m², about 45 g/m² or about 40 g/m². The Cobb Test may be carried out based on procedures described in TAPPI T441 om-20 “Water Absorptiveness of Sized (Non-Bibulous) Paper and Paperboard (Cobb Test)”. The Cobb₃₀ value is based on a sample testing time of 30 seconds.

The composite material may have an improved liquid absorption capacity compared to a similar material that does not contain the thermoset biopolymer derived from separated keratin fibre cellular components. For example, the composite keratin material may have a w/w liquid absorption capacity of about 25 to 60%, about 30 to about 55%, about 35 to 50% or about 40 to 45%. In some embodiments, the composite keratin material comprises the thermoset biopolymer and the synthetic fibre (e.g. a PET fibre) and has a w/w liquid absorption capacity of about 25 to 60%, about 30 to about 55%, about 35 to 50% or about 40 to 45%.

The composite material may have an improved liquid wicking rate compared to a similar material that does not contain the thermoset biopolymer derived from separated keratin fibre cellular components.

The aforementioned values for improved properties are provided to exemplify the invention. Those persons skilled in the art will appreciate that different values may be achieved based on the other components of the composite keratin material. For example, a composite keratin material prepared with a synthetic fibre having a high tensile strength will inherently have a higher tensile strength than a composite keratin material prepared with a lower tensile strength material. Advantageously, the keratin material prepared with the thermoset biopolymer and a synthetic fibre having a high tensile strength may have an improved tensile strength and/or other improved property relative to the same material prepared without the thermoset biopolymer.

Without wishing to be bound by theory, it is believed subjecting the separated keratin fibre cellular components to elevated temperature and elevated pressure causes the cellular separated keratin fibre components to form a film, i.e. an at least partially continuous layer. The degree to which the cellular components have formed a film may be measured by measuring the amount of air that can pass through the material. For example, the composite keratin material may have an air resistance of at least about 1 s/1000 mL, e.g., at least about 20 s/100 mL, about 30 s/100 mL, about 40 s/100 mL, about 50 s/100 mL, about 60 s/100 mL, about 70 s/100 mL, about 80 s/100 mL, about 90 s/100 mL, about 100 s/100 mL, about 110 s/100 mL, about 120 s/100 mL or about 130 s/100 mL. In some embodiments, the composite keratin material has an air resistance of at least about 50 s/100 mL. The air resistance may be measured by conventional techniques known in the art, such as (“Air resistance of paper (Gurley method)”, Test Method T 460 om-21.

The composite keratin material according to the present invention may be useful in various applications, including but not limited to, as a paper material, a fabric, an artificial leather or plastic substitute. Those persons skilled in the art will appreciate that the composition of, and process for manufacturing, the composite of the invention may be modified according to the requirements of such applications.

For example, the composite keratin material comprising the thermoset biopolymer and the synthetic fibre may be useful as a fabric. In some embodiments, the fabric is a non-woven fabric. In this use, the thermoset biopolymer may act as a binder in a non-woven fabric comprising synthetic fibres. Advantageously, in this use the composite keratin material may be used as a partial or complete substitute for chemical binder(s) conventionally used to prepare a non-woven fabric comprising synthetic fibres.

As a plastic substitute, the composite keratin material may be useful in any application in which an oil-based plastic is used, e.g. as a packaging material. For this purpose, the formulation of the composite keratin material may be varied to provide a material with properties suitable for the intended application, e.g., as a soft, flexible material or a rigid material. For example, a rigid composite keratin material may be useful as a solid packaging component, e.g., a tray for food products. Advantageously, the composite keratin material may be formed into various shapes. In some embodiments, the composite keratin material for use a plastic substitute comprises the thermoset biopolymer and a cellulose material.

The following non-limiting examples are provided to illustrate the present invention and in no way limit the scope thereof.

### EXAMPLES

### Example 1 Paper Composites of Varying Composition

Wool cells were added into wood fibre stock on a weight basis of 10%, 45% and 70%.

### Methods and Materials

Sheets of separated wool cells (prepared according to example 1a in WO 2020/080961 A1 as provided below) were supplied by Lincoln Agritech. Two grades of softwood, unbleached Kraft dry-lap pulps were sourced from Oji Fibre Solutions; fibre-cement grade (FCP) and generic standard market Kraft.

Separated wool cells were prepared using the following procedure as described in WO 2020/080961 A1:
in a 12 L vessel, premix a 10 L solution of 1.5 g/L sodium metabisulfite and 0.5 g/L citric acid and heat to 65° C.;
adjust the premix pH to 8.5 with dilute sodium hydroxide;
add 5% on mass of Protex 6L (a bacterial alkaline protease derived from a selected strain of Bacillus licheniformis);
add 450 g of clean chopped wool to the solution and immerse for 8 hours at pH 8.5 and 65° C.; add 1% on mass of Protex 6L to the vessel and leave fully immersed for a further 16 hours at pH 8.5 and 65° C.;
mix the slurry in the vessel using high shear for 30 minutes with 55 mm diameter;
mixing head at about 13000 rpm using an open tooth rotor appropriate for fibrous material;
transfer the mixture to a mesh filter and sieve through a 63 micron screen;
rinse with water;
freeze dry the retentate;
loosen the resulting sheet of dried wool cellular components in a food processor.

### Wool Refining

A 40 gram sample of the wool sheet material was refined in a Wiley mill equipped with a 2.0 mm φ hole screen for approximately 2.0 minutes. After refining, the resultant wool cell material was light and fluffy in nature and a sweet odour was apparent. Moisture content of the refined wool cells was approximately 12%.

### Sample Preparation

Refined wool cells and softwood pulp samples were diluted to 1.2% consistency in demineralised water and soaked overnight. Slurries were then disintegrated at 3000 rpm for 20 minutes using a standard British disintegrator as specified in TAPPI standard T205. Slurries were then further diluted to 0.3% consistency in demineralised water in plastic buckets. Consistencies were confirmed by filtration of slurries through Whatman 113 filters, on a 105° C. oven-dried (OD) basis.

### Pulp Blending

Slurries were blended, based on the pre-determined consistencies, to yield 159 mm diameter handsheets on a Messmer sheet former at a target basis weight of 100 gsm, as per T205 sp-02. The weight ratios of wool cells to wood pulp within the slurry mixtures were: 10:90, 45:55, 70:30 respectively. Control sheets with zero wool cells content (0:100) were used as a basis for comparison.

The appropriate amounts of slurry to yield single handsheets were pre-weighed into plastic jugs and transferred to the handsheet maker to form sheets (five sheets per set). Handsheets were couched (transferred to blotters), pressed, dried and conditioned as per T205 sp-02 and T402 sp-08.

### Hot Pressing of Handsheets

Hot-pressing can be used to modify the properties of the fibre-based sheet, including strength. Handsheets were hot-pressed either when dry or when wet, immediately after forming and couching, at 160° C. for 3 minutes at 900 kN (optimum loading for the Siempelkamp hot press), i.e. 45 MPa pressure for the 200 cm² sheets. To examine the effect of sulfite, sodium sulfite was added to the pulp suspension during sheetmaking or by spraying it directly onto the sheet surface after sheetmaking for comparison. Outcomes were compared to sheets that had received regular pressing and drying after sheetmaking (i.e. not hot-pressed).

### Paper Testing

Conditioned handsheets were tested for grammage, thickness (caliper), density/bulk, tensile strength and air resistance. Relevant standards are listed below:
“Physical testing of pulp handsheets”, Test Method T 220 sp-21
“Tensile properties of paper and paperboard”, Test Method T 494 om-06
“Air resistance of paper (Gurley method)”, Test Method T 460 om-21
“Determination of equilibrium moisture in pulp, paper and paperboard for chemical analysis”, Test method T 550 om-03.

### Results

### Sheetmaking Observations

Handsheets with wool cells contents above 45% were difficult to remove from the sheet wire and required considerable rewetting; retention during sheetmaking was, on average, 94%.

### Handsheet Properties.

Physical properties of handsheets are shown in Table 1. Sheets containing wool cells were bulkier and of lower density compared to the control. Progressively adding wool cells to wood fibre was detrimental to sheet strength (tensile index), presumably due to the lack of inter-fibre bonding between the two different fibre types.

TABLE 1
Handsheet physical properties (23° C., 50% RH).

| | Control (100% | | | 10% wool cells:90% | | | 45% wool cells:55% | | | 70% wool cells:30% | | |
|---|---|---|---|---|---|---|---|---|---|---|---|---|
| | wood pulp) | | | wood pulp | | | wood pulp | | | wood pulp | | |
| | | Dry hot | Wet hot | | Dry hot | Wet hot | | Dry hot | Wet hot | | Dry hot | Wet hot |
| | dry | press | press | dry | press | press | dry | press | press | dry | press | press |
| grammage (g/m²) | 107 | 103 | 103 | 109 | 104 | 104 | 122 | 116 | 102 | 116 | 110 | 101 |
| grammage, oven | 99 | 95 | 95 | 101 | 96 | 96 | 112 | 105 | 92 | 107 | 99 | 91 |
| dry (g/m², 105° C.) | | | | | | | | | | | | |
| caliper (μm) | 218 | 107 | 141 | 228 | 109 | 121 | 310 | 98.1 | 127 | 344 | 94.8 | 117 |
| density (g/cm³) | 0.491 | 0.963 | 0.730 | 0.478 | 0.954 | 0.860 | 0.393 | 1.182 | 0.803 | 0.337 | 1.160 | 0.863 |
| bulk (cm³/g) | 2.0 | 1.04 | 1.37 | 2.1 | 1.05 | 1.16 | 2.5 | 0.85 | 1.25 | 3.0 | 0.86 | 1.16 |
| tensile index (N · m/g) | 13.56 | 16.16 | 27.76 | 11.43 | 19.08 | 48.17 | 4.88 | 50.97 | 47.88 | 1.85 | 48.28 | 143.72 |
| air resistance (s/300 | 1.2 | 25.0 | 20.4 | 2.2 | 45.6 | 74.0 | 5.0 | — | 486 | 5.6 | — | — |
| mL, 1.22 kPa) | | | | | | | | | | | | |

In addition, New Zealand Crossbred wool fibre was also incorporated into a lower coarseness (and therefore higher fibre count per gram) unbleached Kraft pulp at 45% wool fibre: 55% wood weight ratio to see if tensile strength could be enhanced. Limited testing showed these alternative sheets had a tensile strength of 6.23N·m/g as opposed to 4.88N·m/g for the equivalent FCP-wool cells based furnish.

Comparison of the physical appearance of the sheets showed lighter colourisation as the quantity of wool fibre progressively increased relative to the FCP wood fibre mix. Upon handling, sheets became flimsy in texture as more wool fibre was included in the overall fibre mix (in keeping with bulk test results).

### Tensile Strength

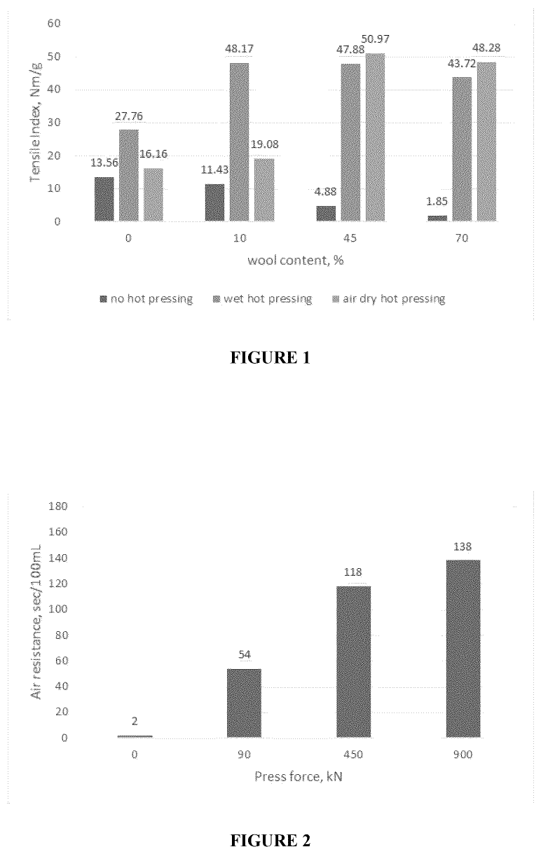

Despite the sheet deformation brought about by wet hot-pressing, the tensile strength of sheets could be significantly improved compared to regular sheetmaking procedures (FIG. 1). Regardless of wool cells content, the gains in tensile strength after wet hot-pressing were similar for mixed sheets; however, this was not the case for the hot-pressed air-dried sheets.

### Example 2—Hot-Pressing Optimisation and Effects of Sodium Sulfite Addition on Wool Cells Paper Composites

The focus of these experiments was to examine the effects of sodium sulfite as an additive and varying the pressing force on paper property outcomes; of particular interest was the degree of air, water and grease barrier properties achievable. Handsheets were hot pressed wet, immediately after forming and couching, at 160° C. for 3 minutes under varying press loads (90KN-900KN). Sulfite addition included adding it to the pulp suspension during sheetmaking or spraying it directly onto the sheet surface after sheetmaking for comparison. All handsheets were made from a blend of 45% wool cells and 55% wood fibre. Outcomes are compared to previous sheets containing wool cells that had received regular pressing and drying (i.e. not hot-pressed) and sheets made from 100% wood fibre.

### Wool Cells Refining

The preparation of wool cells material was as described in Example 1.

### Handsheet Making

Handsheets were formed based on Appita standard method AS/NZS 1301.203s:2017-“Forming handsheets for physical testing of pulp” but instead of the regular pressing and drying procedures, the sheets were hot-pressed immediately.

### Hot-Pressing Method

Handsheets were hot pressed between two metal plates using the Siempelkemp press at 160° C. for 3 minutes using three wet pressing forces: 90 kN, 450 kN and 900 kN (equivalent to sheet pressures of 4.5 Mpa, 22.5 Mpa and 45 Mpa, respectively).

### Paper Testing

Conditioned handsheets were tested for grammage, thickness (caliper), density/bulk, tensile strength, burst strength, short span compression strength (SCT) following the methods listed in Example 1. In addition, testing for air, water and grease resistance was performed following the procedures below.

### Air Resistance

Preliminary testing indicated it was not possible to test air resistance using the manual Gurley porosimeter that was used previously as some sheet permeability values extended beyond the machine's measuring range and testing proved slow and laborious. Instead, an automatic L&W Air Permeance Tester that suits a variety of paper products and can test highly impermeable materials with ease was used and is in accordance with the most common measuring methods (“Air resistance of paper (Gurley method)”, Test Method T 460 om-21). Relative air resistance values were reported as see/100 mL.

### Water Resistance

The Cobb Test was carried out based on procedures described in TAPPI T441 om-20 “Water Absorptiveness of Sized (Non-Bibulous) Paper and Paperboard (Cobb Test)”. Due to the nature of the sample material, the period of time for the test was reduced to 30 seconds and the water charge was halved to ensure accurate testing. Cobb₃₀ water absorbency is quoted in g/m².

### Grease Resistance

Grease resistance was tested following ISO 16532-1: 2008 (en) “Paper and board-Determination of grease resistance—Part 1: Permeability test”.

Following ISO 5634-1986 E “Paper and board-Determination of grease resistance”, show-through and break-through times in seconds (mean and range) for handsheets were reported as well as stain area (mm²) after 24 hours to gauge long term grease resistance.

### Results—Effect of Wet Pressing Force on Sheet Properties

### Visual Observations

As observed in previous Examples, there was deformation of the sheet surface after wet hot-pressing; increasing the pressing force progressively led to more blistering of the sheet surface.

Table 2 summarises the properties of the sheets wet hot-pressed at forces ranging from 90 kN to 900 kN.

TABLE 2
Handsheet physical properties (23° C., 50% RH) for wet,
hot-pressed (0 kN-900 kN) 45% wool cells:55% wood pulp sheets.

| | 0 kN | 90 kN | 450 kN | 900 kN |
|---|---|---|---|---|
| grammage (g/m²) | 111 | 96 | 94 | 102 |
| grammage, oven dry (g/m², | 102 | 89 | 87 | 92 |
| 105° C.) | | | | |
| caliper (μm) | 274 | 118 | 110 | 127 |
| density (g/cm³) | 0.403 | 0.815 | 0.858 | 0.803 |
| bulk (cm³/g) | 2.48 | 1.23 | 1.17 | 1.25 |
| burst index (kNm/g) | 1.1 | 3.4 | 4.0 | 4.6 |
| SCT index (Nm · g) | 4.4 | 46.0 | 37.3 | 33.9 |
| tensile index (N · m/g) | 7.10 | 37.6 | 50.4 | 47.9 |

Compared to the un-pressed sheet, wet hot-pressing generally doubled the sheet density of the 45% wool cells: 55% wood fibre sheets, which remained relatively constant despite the differing pressing force used. The sheet density of the 900 KN sheet was lower than expected but could be due to the difficulty in measuring caliper accurately on the non-uniform sheet surfaces. Upon wet hot-pressing at 90 kN, sheet tensile strength improved significantly, and further strength gains were possible if the force was increased to or above 450 kN. Bursting strength and short span compression (SCT) are also considered important attributes for some paper products so were tested for sample points where sufficient test material was available. Hot-pressing and increasing pressing force resulted in higher burst indices compared to the control sheet. Like other properties, upon hot-pressing, the short span compression index improved greatly.

### Barrier Properties

Because paper is composed of a randomly felted layer of fibre, the structure has a varying degree of porosity. Thus, the ability of fluids, both liquid and gaseous, to penetrate the structure is a property both highly significant to the use of paper and capable of being widely varied by the conditions of manufacture. The air, water and grease resistance of the 45% wool cells: 55% wood fibre handsheets are compared in the following sections.

### Air Resistance

FIG. 2 shows the effect of increasing pressing force on air resistance. It is believed the air resistance is influenced by a sheet's internal structure, which is in turn governed by compaction, fines material, etc. Increasing wet pressing force progressively raised air resistance up to 450 kN at which point it had begun to level off.

### Water Resistance

The Cobb test is routinely used to test the ability of a paper material to resist the ing procedure determines the amount of water absorbed by a paper sheet in a specified time under standardised conditions. If a fibre material absorbs too much water, the paper formed from it may have difficulty maintaining strength and integrity.
A high Cobb value means a substrate has greater ability to absorb and retain water.
A low Cobb value means the substrate has greater resistance to penetration and retention of water.

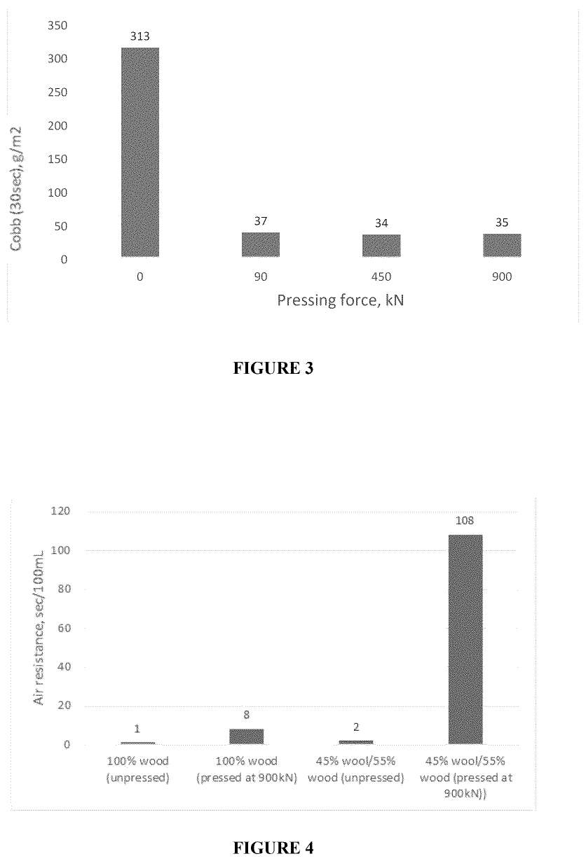

The effect of pressing force on Cobb value for the 45% wool cells: 55% wood fibre handsheets is shown in FIG. 3. Notably, wet hot-pressing substantially reduced water absorption with Cobb₃₀ decreasing from 313 g/m² for unpressed sheets to below 40 g/m² after pressing.

### Grease Resistance

Grease resistance is tested by applying a standard oil sample to the sheet surface and is described by two characteristics: “show-through” time (visual penetration) and “break-through” time (actual penetration). Show-through refers to the visual appearance of grease stain on the underside of the sample while break-through is the visual staining of copy paper (as specified) beneath the sample.

Show-through and break-through times (mean and range) for handsheets made from 45% wool cells: 55% wood fibre mix and 100% wood fibre are summarised in Table 3. The results demonstrate that wet pressing reduced show-through times (i.e. lowered grease resistance). Break-through times were also reduced, which is what is typically observed for many papers using this test.

TABLE 3
Mean and range “show-through” and “breakthrough” times for wool
cells:wood fibre mix handsheets and 100% wood fibre handsheets.

| | Wet pressing | Mean | |
|---|---|---|---|
| | (900 kN, 160° | time | Range |
| Sample composition | C., 3 mins) | (seconds) | (seconds) |
| Show-through | | | |
| 100% wood fibre | − | 488 | |
| 100% wood fibre | + | 188 | |
| 45% wool cells:55% wood | − | 625 | 536-783 |
| 45% wool cells:55% wood | + | 100 | 60-120 |
| Break-through | | | |
| 100% wood fibre | − | 1555 | |
| 100% wood fibre | + | 1260 | |
| 45% wool cells:55% wood | − | 1644 | 1406-1796 |
| 45% wool cells:55% wood | + | 618 | 423-723 |

To gauge long term grease penetration, the stain area (mm²) under the sample after 24 hours can be measured. Stain areas for the unpressed and pressed sheet at 900 KN were 1541 mm² and 1335 mm², respectively, and suggest pressing has a limited negative effect on long term grease resistance.

### Results—Effect of Sodium Sulfite on Sheet Properties

The effect of sodium sulfite addition on 45% wool cells: 55% wood sheet properties is shown in Table 4. Additive was applied either directly onto the surface of the handsheets after couching or added to the papermaking solution prior to sheetmaking. All sheets were wet hot-pressed at 900 KN, 160° C. for 3 minutes.

TABLE 4
Handsheet physical properties (23° C., 50% RH)
for wet, hot-pressed (900 kN, 160° C., 3 minutes)
45% wool cells:55% wood pulp sheets containing additive.

| | | Additive | Additive in | Additive in |
|---|---|---|---|---|
| | | sprayed on | 1 w/w % | 1 w/w % |
| | No | surface | solution | solution |
| | additive | (4 mL) | (4 mL) | (666 mL) |
| grammage (g/m²) | 102 | 98 | 102 | 98 |
| grammage, oven dry | 92 | 88 | 92 | 88 |
| (g/m², 105° C.) | | | | |
| caliper (μm) | 127 | 130 | 141 | 135 |
| density (g/cm³) | 0.803 | 0.752 | 0.721 | 0.726 |
| bulk (cm³/g) | 1.25 | 1.33 | 1.39 | 1.38 |
| burst index (kNm/g) | 1.1 | 2.9 | 3.2 | 3.9 |
| SCT index (Nm · g) | 33.9 | 35.5 | 39.1 | 39.8 |
| tensile index (N · m/g) | 47.9 | 50.4 | 58.6 | 48.9 |

The addition of sulfite into wet pressed 45% wool cells: 55% wood fibre handsheets had a positive effect on grease resistance, raising show-through and break-through times from 100 to 513 seconds and 618 to 1108 seconds, respectively (Table 5). Stain area was also reduced for wool cells:wood fibre sheets containing additive (938 mm²) compared to those without additive (1335 mm²).

TABLE 5
Mean and range “show-through” and “break-through”
times for wool cells/wood fibre mix handsheets.

| | Wet pressing | | Mean | |
|---|---|---|---|---|
| | (900 kN, 160° | | time | Range |
| Sample composition | C., 3 mins) | Additive | (seconds) | (seconds) |
| Show-through | | | | |
| 45% wool cells/ | + | − | 100 | 60-120 |
| 55% wood | | | | |
| 45% wool cells/ | + | + | 513 | 363-694 |
| 55% wood | | | | |
| Break-through | | | | |
| 45% wool cells/ | + | − | 618 | 423-723 |
| 55% wood | | | | |
| 45% wool cells/ | + | + | 1108 | 1054-1161 |
| 55% wood | | | | |

### Main Outcomes

The wet hot-pressing method used in this example provided the following outcomes:
Raised sheet tensile strength; at 90 kN pressing force, sheet tensile strength improved from 7 Nm/g to 37 Nm/g, and further strength gains up to 50 Nm/g were possible if force was increased to 450 kN and above.
Progressively increased air resistance from 2 sec/100 mL for unpressed sheets up to 138 sec/100 mL for the sheets pressed at 900 kN.
Lowered Cobb₃₀ values significantly; Cobb₃₀ decreased from 313 g/m² for unpressed sheets to below 40 g/m² after pressing at all levels of pressing force.
Had a negative effect on both short- and long-term grease resistance.

Further, the addition of sodium sulfite provided the following outcomes:
The method of sulfite application and quantity did not alter density, tensile strength or air resistance.
Did not improve water resistance of wet pressed sheets.
Had a positive effect on grease resistance, raising show-through and break-through times from 100 to 513 seconds and 618 to 1108 seconds, respectively.

### Results—Effect of Adding Wool Cells to Wood Fibre on Sheet Properties

The effect of adding wool cells to wood fibre on sheet properties for unpressed and wet hot-pressed (900 KN, 160° C., 3 minutes) sheets is shown in the tables and figures below.

TABLE 6
Handsheet physical properties (23° C., 50% RH)
for unpressed and wet hot-pressed (900 kN) 100% wood
pulp sheets and 45% wool cells:55% wood fibre sheets.

| | | | Unpressed | Pressed |
|---|---|---|---|---|
| | Unpressed | Pressed | 45% wool | 45% wool |
| | 100% | 100% | cells:55% | cells:55% |
| | wood | wood | wood | wood |
| Pressing force | 0 kN | 900 kN | 0 kN | 900 kN |
| grammage (g/m²) | 95.28 | 105.46 | 110.46 | 102 |
| grammage, oven dry (g/m², | 87.65 | 97.03 | 101.63 | 92 |
| 105° C.) | | | | |
| caliper (μm) | 219 | 129 | 274 | 127 |
| density (g/cm³) | 0.523 | 0.815 | 0.403 | 0.803 |
| bulk (cm³/g) | 2.08 | 1.23 | 2.48 | 1.25 |
| burst index (kNm/g) | 0.6 | 2.1 | 1.1 | 4.6 |
| SCT index (Nm · g) | 13.6 | 17.2 | 4.4 | 33.9 |
| tensile index (N · m/g) | 21.0 | 24.5 | 7.10 | 47.88 |

Adding wool cells to wood fibre sheets that were unpressed tended to lower strength; however, if both sheets were wet hot-pressed, the wool cells increased strength-related properties substantially (Table 6).

Unpressed 100% wood fibre sheets and 45% wool cells: 55% wood fibre mix sheets had similar air resistance; however, upon wet pressing under the same conditions, the air resistance of sheets comprising wool cells increased dramatically in contrast to the 100% wood fibre sheet (FIG. 4).

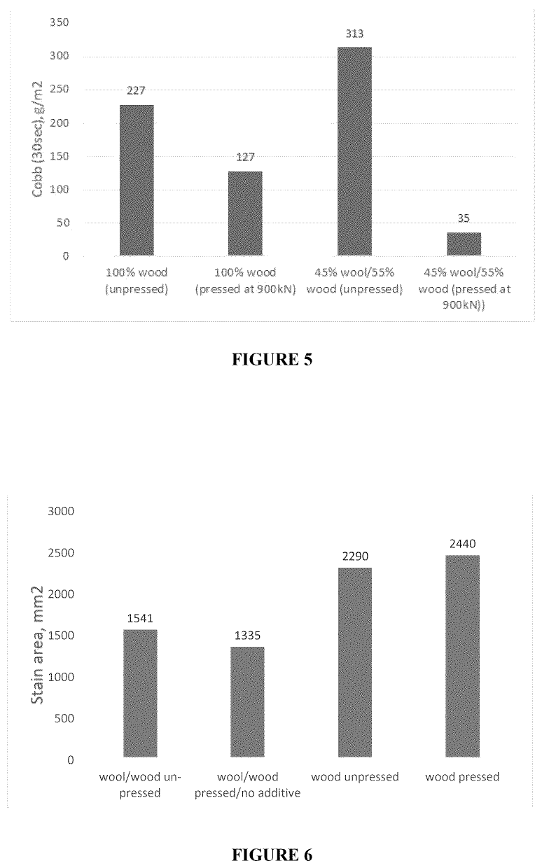

In an unpressed state, adding wool cells to wood fibre raised Cobb₃₀ from 227 g/m² to 313 g/m². However, upon wet pressing both types of sheet became more water resistant (FIG. 5), particularly the sheets comprising wool cells.

### Main Outcomes

Compared to 100% wood fibre handsheets:
adding wool cells to wood fibre without wet hot pressing caused decreases in sheet tensile strength. However, upon wet hot pressing, tensile strength more or less doubled for the handsheets comprising wool cells.
the wool cells:wood fibre mix sheets were less water resistant when unpressed, but the opposite was true after wet pressing. The wool cells:wood fibre mix sheets displayed superior short- and long-term grease resistance.

### Example 3—Preparation of Wool Cells/PET Composite

Four wetlaid nonwoven fabrics were prepared from PET fibre (6 mm, 1.7 dtex) and separated wool cells (prepared according to WO 2020/080961 A1) using a wetlaid process (Table 7). The PET fibres and separated wool cells were blended with processing aids (Pluronic F-108—dispersing agent, 1 w/w % and Tetronic 90R4—defoaming agent, 1 w/w %) to provide a homogenous slurry. The slurry was deposited on a surface and the water was removed to provide a dry web. A 1 w/w % Na₂SO₃ solution was added dropwise to the dry web until uniform saturation of the web was achieved. The saturated web was hot-pressed at 160° C. for 3 minutes and a pressure as high as physically possible through hydraulic action.

TABLE 7
PET/wool cells weight ratio of composite materials

| Sample | Total density (g/m²) | PET/wool cells ratio |
|---|---|---|
| 3A | 180 | 60:40 |
| 3B | 180 | 80:20 |
| 3C | 180 | 90:10 |
| 3D | 180 | 100:0 |

### Results

Samples showed increasing stiffness and browning with increasing wool cells content.

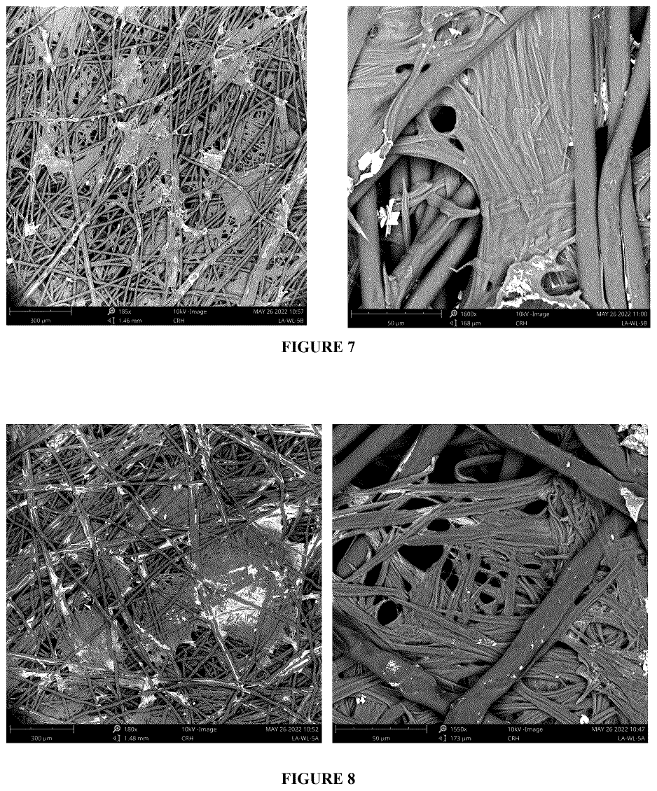

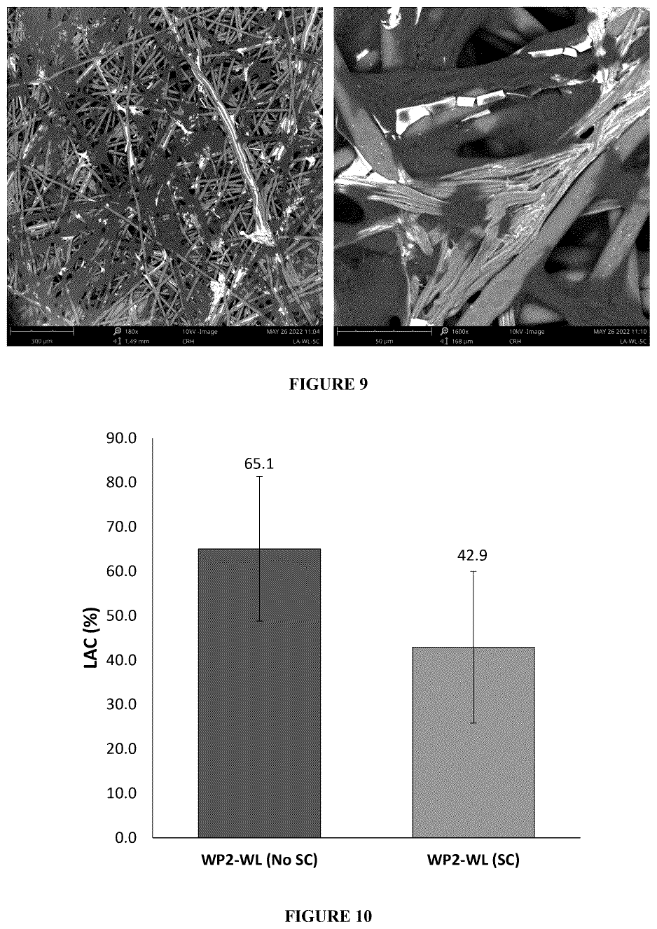

SEM images of Samples 3A-3C are shown in FIGS. 7-10, respectively. SEM analysis of these samples showed increasing melding with increasing wool cells content. The fibres in Sample 3D did not bind when hot-pressed, resulting in a weak fabric. SEM analysis was not conducted on Sample 3D to avoid sensor damage due to the loose fibre content.

Samples hot-pressed without wool cells content were weak, demonstrating that the binding is linked to the presence of melded wool cells. The wool cells acted effectively as a binder and substituted for a chemical binder that would be used in producing synthetic nonwoven textiles.

### Example 4—Comparison of PET Materials with and without Wool Cells

Two examples of wetlaid nonwoven fabrics were prepared from PET fibre (6 mm, 1.7 dtex), 10 w/w % polypropylene/polyethylene biocomponent fibre (Bico) and separated wool cells (prepared according to WO 2020/080961 A1) using a wetlaid process (Table 8). The 10% bicomponent fibre was included to enable handling of PET sample without separated wool cells post hot pressing process. Processing aids (Pluronic F-108-dispersing agent, 1 w/w % and Tetronic 90R4—defoaming agent, 1 w/w %) added to slurry to aid dispersion and homogeneity of slurry/fabric.

TABLE 8

| | Total areal | SC areal | |
|---|---|---|---|
| | density | density | |
| Sample | (g/m²) | (g/m²) | Composition |
| 4A | 180 | 0 | 90% PET/10% Bico |
| 4B | 180 | 72 | 50% PET/40% SC/10% Bico |

### Results

The addition of wool cells resulted in a reduction (although not significantly different (P>0.05)) of the liquid absorption capacity of the melded wetlaid fabric (FIG. 10). Without wishing to be bound by theory, it is thought this reduction could be due to the closing of the pore structure and formation of film-like structure during SC melding.

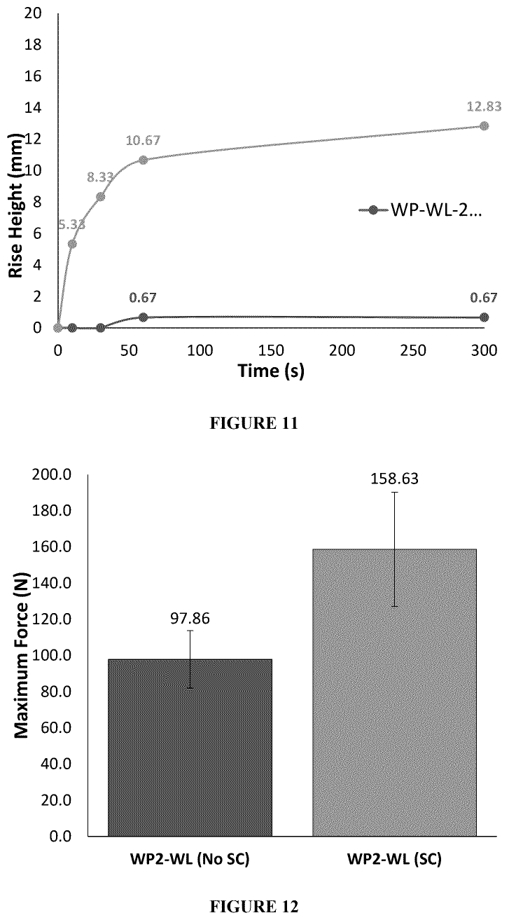

The addition of wool cells resulted in a large increase in the fabric wicking rate (FIG. 11). This demonstrates the wicking ability of the melded separated keratin fibre cellular component of the fabric.

The inclusion of wool cells and subsequent melding resulted in a 62% increase in tensile strength of the fabric (FIG. 12). The increase is attributed to the melding process, with the formation of additional physical bonds between fibres within the fabric.

These results demonstrate that melding separated wool cells can bond fibres together and have a significant impact on fabric properties

### Example 5—Keratin Film

A mixture of wool cells in a 2:1 w/w liquor ratio (1 w/w % sodium sulfite solution in water:wool cells) were pressed into a solid film at 160° C. and 780 Bar (11318 psi) for 3 minutes. Materials were pressed in a steel die having a contact area of 1096 mm².

The resulting film was rigid and transparent and demonstrated that the individual wool cells had melded to create a single continuous phase of material. The material did not change dimension or appearance on immersion in water and remained physically robust.

### Example 6—Plastic Substitute

The materials made using the methods of examples 1, 2 and 5 were used as physical barriers and packaging materials to contain a range of items. These materials performed as effective substitutes for conventional synthetic plastic, for example PET or PE film.

### Example 7—Artificial Leather

The materials made using the methods of examples 1 and 2 were used as surface coverings and inner and outer layers of bags and clothing materials. The protein rich composition of the material (due to the high keratin content) gave a sensorial feel similar to leather. The robustness of the materials allowed them to act as a substitute for natural leather and as a result the materials performed as a synthetic leather.

### Example 8—Glycerol Plasticiser

Cellulose/wool composite paper sheets made using the method of example 1 were treated by immersing in aqueous glycerol solution (1-50%) blotted dry and then pressed according to the method described in example 2. In some cases, the sheets were washed in water or dilute sulfuric acid (2 w/w %) after pressing, dried in a dessicator and pressed again with water. Washing and drying the sheets made them less sticky compared with unwashed sheets. Glycerol-melded materials (whether washed or not) were observed to be more flexible than water-melded composites.

### Example 9—Preparation of Multi-Layered Composite

Sheets made using the method of example 1 were layered in 2 to 5 sheets and hot pressed as described in example 2 to form materials with caliper measured thickness greater than 200 μm.

Optionally, the sheets were treated with glycerol and then layered on top of each other and pressed as described in example 2. The best results in terms of melding and leather-like hand-feel were obtained with the following conditions: 3×70% SC hand sheets were pre-treated with 50 w/w % v/v glycerol and blotted dry, then hot pressed at 180° C. and 50 kN pressure for 30s. The hot pressing process was repeated up to 3 times.

The products obtained by these methods were observed to be more flexible than water-melded composites, and had good hand feel (leather-like). These structures can be used to form artificial leather materials as described in example 7.

### Example 10—Natural or Synthetic Oil

Hand sheets made according to the method of example 1 were immersed in oil and blotted or lightly brushed with oil before melding. Silicone oil and coconut oil were tested. Samples were melded under conditions as described in example 2, including 180° C. at 50 kN pressure for 30 seconds.

The oil melded materials appeared to be less translucent than glycerol or water melded composites and more flexible than water-melded materials.

It is not the intention to limit the scope of the invention to the abovementioned examples only. As would be appreciated by a skilled person in the art, many variations are possible without departing from the scope of the invention as set out in the accompanying claims.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

1. A thermoset biopolymer derived from separated keratin fibre cellular components.

2. The thermoset biopolymer of claim 1, where the thermoset biopolymer is obtained by subjecting the separated keratin fibre cellular components to elevated temperature and elevated pressure.

3. A composite keratin material comprising a thermoset biopolymer, wherein the thermoset biopolymer is derived from separated keratin fibre cellular components.

4. The composite keratin material of claim 3, wherein the thermoset biopolymer is obtained by subjecting the separated keratin fibre cellular components to elevated temperature and elevated pressure.

5. The composite keratin material of claim 3 or 4, wherein the composite keratin material further comprises cellulose fibres or polyethylene terephthalate (PET).

6. A process for preparing a composite keratin material comprising the steps of:
a) providing a mixture comprising separated keratin cellular fibre components, and
b) subjecting the mixture to elevated temperature and elevated pressure such that the separated keratin cellular fibre components form a thermoset biopolymer to provide the composite keratin material.

7. The process of claim 6, wherein the elevated temperature is at or above the glass transition temperature of the separated keratin fibre cellular components.

8. The process of claim 6 or 7, wherein the elevated temperature is at least about 140° C.

9. The process of any one of claims 6 to 8, wherein the elevated pressure is at least about 40 kN, optionally at least about 90 kN.

10. The process of any one of claims 6 to 9, wherein mixture is subjected to elevated temperature and elevated pressure by hot-pressing.

11. The process according to any one of claims 6 to 10, wherein the mixture in step (a) is provided as a sheet.

12. The process according to any one of claims 6 to 11, wherein mixture in step (a) is provided as a layer of two or more sheets, optionally a layer of 2 to 10 sheets or 2 to 5 sheets.

13. The process according to claim 11 or 12, wherein each sheet is formed by pressing a slurry comprising separated keratin cellular fibre components, optionally wherein each sheet is couched, pressed and dried.

14. The process of any one of claims 6 to 13, wherein the mixture further comprises water.

15. The process of any one of claims 6 to 14, wherein the mixture further comprises an additive selected from the group consisting of a reducing agent, a plasticiser, an oil and a combination of any two or more thereof.

16. The process of claim 15, wherein the reducing agent is selected from the group consisting of a sulfite, a metabisulfite, a sulfide, a thioglycolate, cysteine and a combination of any two or more thereof.

17. The process according to claim 15 or 16, wherein the plasticiser is selected from glycerol or polypropylene glycol, optionally wherein the glycerol is an aqueous glycerol solution, a 1-50 w/w % aqueous glycerol solution, a 10-50 w/w % aqueous glycerol solution or a 50 w/w % aqueous glycerol solution.

18. The process according to any one of claims 15 to 17, wherein the oil is a natural oil (e.g. a vegetable oil, such as almond oil, avocado oil, coconut oil, palm oil, peanut oil, canola oil, safflower oil, sesame oil, soyabean oil, sunflower oil, grapeseed oil, or an animal-derived oil,) or a synthetic oil (e.g. silicone oil).

19. The process according to any one of claims 15 to 18, wherein the mixture in step (a) is provided as a sheet or layer of sheets and the additive is applied to the sheet before step (b), and optionally wherein the additive is applied by immersing the sheet or layer of sheets in the additive, brushing the additive onto the sheet or layer of sheets or spraying the additive on the sheet or layer of sheets, optionally wherein the additive is applied to each sheet before forming the layer of sheets.

20. The composite keratin material of any one of claims 3 to 5 or the process of any one of claims 6 to 19, wherein the composite keratin material has one or more of the following properties:
i) a tensile strength of at least about 30 Nm/g,
ii) an air resistance of at least about 10 s/100 mL,
iii) a Cobb₃₀ value below about 100 g/m².

21. The composite keratin material or the process of claim 20, wherein the composite keratin material has at least two of properties i) to iii).

22. The composite keratin material of any one of claims 3 to 5, 20 and 21 or the process of any one of claims 6 to 21, wherein the keratin fibre cellular components are obtained from a source selected from the group consisting of wool, hair, fur, feathers and a combination of any two or more thereof.

23. The composite keratin material of any one of claims 3 to 5 and 20 to 22 or the process of any one of claims 6 to 22, wherein the keratin fibre cellular components are obtained from wool.

24. The composite keratin material of any one of claims 3 to 5 and 20 to 23 or the process of any one of claims 6 to 23, wherein the composite keratin material further comprises a cellulose material.

25. The composite keratin material or the process of claim 24, wherein the cellulose material is derived from a plant or plant parts.

26. The composite keratin material or the process of claim 24 or 25, wherein the composite keratin material comprises keratin fibre cellular components and the cellulose material in a weight ratio of about 5:95 to about 95:5.

27. The composite keratin material of any one of claims 3 to 5 and 20 to 24 or the process of any one of claims 6 to 24, wherein the composite keratin material further comprises a synthetic fibre.

28. The composite keratin material or the process of claim 27, wherein the synthetic fibre comprises a polymer selected from the group consisting of a polyester (e.g. a polyethylene terephthalate, PET), a polyacrylic, a polychloroprene (e.g. neoprene), a polyolefin, a polyurethane (e.g. spandex), a polyamide (e.g. nylon) and a combination of any two or more thereof.

29. The composite keratin material of any one of claims 3 to 5 and 20 to 28 or the process of any one of claims 6 to 28, wherein the composite keratin material has a tensile strength of at least about 35 Nm/g, about 36 Nm/g, about 37 Nm/g, about 38 Nm/g, about 39 Nm/g, about 40 Nm/g, about 41 Nm/g, about 42 Nm/g, about 43 Nm/g, about 44 Nm/g, about 45 Nm/g, about 46 Nm/g, about 47 Nm/g, about 48 Nm/g, about 49 Nm/g, about 50 Nm/g, about 51 Nm/g, about 52 Nm/g, about 53 Nm/g, about 54 Nm/g or about 55 Nm/g.

30. The composite keratin material of any one of claims 3 to 5 and 20 to 29 or the process of any one of claims 6 to 29, wherein the composite keratin material has an air resistance of at least about 20 s/100 mL, about 30 s/100 mL, about 40 s/100 mL, about 50 s/100 mL, about 60 s/100 mL, about 70 s/100 mL, about 80 s/100 mL, about 90 s/100 mL, about 100 s/100 mL, about 110 s/100 mL, about 120 s/100 mL or about 130 s/100 mL.

31. The composite keratin material of any one of claims 3 to 5 and 20 to 30 or the process of any one of claims 6 to 31, wherein the composite keratin material has a Cobb₃₀ value below about 90 g/m², about 80 g/m², about 70 g/m², about 60 g/m², about 50 g/m², about 45 g/m² or about 40 g/m².

32. The composite keratin material of any one of claims 3 to 5 and 20 to 31 or the process of any one of claims 6 to 31, wherein the composite keratin material is in the form of a sheet.

33. The composite keratin material or the process of claim 32, wherein the sheet has a thickness greater than 100 μm, greater than 150 μm, greater than 200 μm or greater than 250 μm.

34. A composite keratin material prepared by a process according to any one of claims 6 to 33.

35. A paper comprising the composite keratin material according to any one of claims 3 to 5 and 20 to 34.

36. An artificial leather comprising the composite keratin material according to any one of claims 3 to 5 and 20 to 34.

37. A fabric comprising the composite keratin material according to any one of claims 3 to 5 and 20 to 34.

38. The fabric of claim 37, wherein the fabric is a non-woven fabric.

39. Use of the composite keratin material according to any one of claims 3 to 5 and 20 to 34 as a plastic substitute.

40. Use of the composite keratin material according to any one of claims 3 to 5 and 20 to 34 as a substitute for a chemical binder in the preparation of a synthetic non-woven textile.
